(19) United States
(12) Patent Application Publication
(10) Pub. No.: US 2003/0171795 A1
(43) Pub. Date: Sep. 11, 2003

(54) Low level laser therapy method and means

(76) Inventors: Richard Walmsley, South Yarra (AU); Patricia Ann Angel, Henley Beach (AU)

Correspondence Address: KLAUBER & JACKSON, 411 HACKENSACK AVENUE, HACKENSACK, NJ, 07601

(21) Appl. No.: 10/367,572

(22) Filed: Feb. 14, 2003

(30) Foreign Application Priority Data
Feb. 14, 2002 (AU) PS 0510

(51) Int. Cl. A61N 1/00

(52) U.S. Cl. 607/88

(57) ABSTRACT

The invention described provides a treatment for edema, lymphedema and extra-cellular fluid with Low Level Laser Therapy (LLLT). The invention in particular will be effective in the treatment of lymphedema that includes the effects of lymph gland damage, disease or removal (LO) following surgery typically associated with cancer treatment or at least provides an alternative to other treatments. In particular the use of LLLT according to the method and apparatus of the invention can effectively treat LO and post surgery LO of a limb associated with the removal of a lymph gland. A method of treatment of the lymphatic system/lymphedema and edema in a mammalian subject includes the step of radiation of the surface of the skin of a mammal in the area of physiological-concern with a low-level infrared laser. The laser is a Class 1 laser arranged to emit a pulsed beam. The laser is applied at discrete points on the surface of the skin for up to 1 minute per point in the area of physiological concern being nodal areas adjacent to an affected limb. The wavelength of the laser emission is between 600 to 1100 nm the laser having pulse widths from 1 nanosecond to 1 second with peak powers from 1 milliwatt to 1000 Watts, average powers from 1 microWatt to 1000 milliWatts at repetition rates from 0.1 to 100 kilohertz. Further, the energy of the laser is delivered at substantially 5 Joules with an energy density of about 1.5 Joules per square centimetre.

This invention relates to the treatment of edema including lymphatic system induced edema wherein the treatment involves Low Level Laser Therapy (LLLT) in particular for the treatment of lymphedema (LO) both primary and secondary. An example is provided in treating post-mastectomy LO with LLLT.

### BACKGROUND

At the molecular level, there are reports that LLLT affects cytochromes of the mitochondrial electron transport chain (Karu 1989), and induces local gradients in energy delivery due to laser speckle resulting in local gradients in cellular heating (Horváth & Donko 1992). At the cellular level, LLLT is reported to stimulate mitogenic activity, adhesion, synthetic activity and viability of fibroblasts (Abergel et al, 1984; Boulton & Marshall 1986; Glassberg et al 1988; Yu et al, 1994; Conlan et al, 1996, Bednarska et al, 1998), although this may only be true for systems that are operating sub-optimally (Abergel et al, 1984). Macrophages were stimulated by LLLT to produce factors that increased or decreased fibroblast proliferation, depending on the wavelength of laser used (Young et al, 1990). LLLT stimulate lymphocytes to proliferate and to become activated, both in vitro and in vivo (Inoue et al 1989; Tadakuma, 1993; Ganju et al, 1999), although again this may only be true in pathological settings, where LLLT ‘primes’ lymphocytes to be more responsive to natural stimulatory products (Smol'yaninova et al, 1991). All of these cell types may have a role to play in resolution of lymphedema.

At the cellular level, it has been suggested that there are stimulatory/protective effects of applying LLLT on endothelial cells and vascular endothelium in situ (Lamuraglia et al 1992). This may involve angiogenic factor production by T-lymphocytes (associated with endothelial cell proliferation; Agaiby et al, 2000), or increased vascular endothelial growth factor (VEGF) production by smooth muscle cells or fibroblasts (Kipshidze et al, 2001). Use of LLLT enhanced endothelial regeneration after damage in animal models (De Scheerder et al, 1998; Kipshidze et al, 1998), and in humans after coronary arterial stent implantation (De Scheerder et al, 2000). The inventors have not seen any reports of LLLT on lymphangiogensis, but proposed that lymphatic vessels will respond similarly to blood vessels, since members of the VEGF family, VEGF-C and -D, stimulate lympangiogensis (Plate, 2001). There are reports of stimulation of local fluid circulation (Horváth & Donko, 1992), and stimulatory effects on lymphatic vessels (Lieviens et al, 1985), probably in response to increased fluid mobility in radiated tissues. There does not seem to be a direct consistent effect of low level laser on lymphatic vessel contractility when laser is applied to the vessels alone (Carati et al, 1998).

Therapeutic application of non-thermal Low Level Lasers using a bio-stimulative or photochemical effect was first proposed in the 1960's by Mester et al for a multitude of neurological, musculoskeletal and soft tissue conditions. In vivo animal trials have noted increased lymphatic motility in wounds treated with LLLT.

There is also evidence that LLLT is useful for stimulating fibroblastic activity (to reduce scar tissue) and for stimulating the immune system (particularly the lymphocytes as well as macrophages).

Upper limb lymphedema (LO) is a common and distressing complication of breast cancer surgery (Browning et al, 1997; Petrek and Heelan, 1998). Reported incidence after surgery is around 5%, increasing to 30% with the administration of adjunctive radiotherapy. It is a chronic and progressive condition in which there is swollen limb deformity, often accompanied by a brawny edema.

Patient discomfort is common with symptoms of limb heaviness, weakness, pain, restricted shoulder mobility, burning pains and elevated skin temperature, obvious deformity, social isolation and psychological morbidity. Traditional treatments for this condition have included compression bandaging, manual lymphatic drainage and extended limb elevation (Foldi and Foldi, 1985). Due to the nature of these treatments, none have been validated with placebo controlled trials.

Also, these treatments are expensive, time consuming and labour intensive (Casley-Smith and Casley-Smith, 1997).

Treatment is expensive in most countries and patients suffer high relapse rates (˜80%) on cessation of treatment. Other treatments, including ultrasound therapy and drug therapy, are also only partly effective, have different latencies, and are again subject to high relapse rates. Hepatotoxicity of chronic benzopyrone therapy has been reported and resulted in the withdrawal of benzopyrone from the Australian and US markets. The cost to the patient of continuing treatment remains a significant burden in most countries, typically delivered in private clinics by allied health personnel, or in a limited manner through Government health centres. Costs are not covered or covered in a limited manner by health insurance.

Secondary LO typically occurs after lymph glands are removed as part of a cancer surgery procedure.

The cancers that typically involve lymph gland removal include breast, prostrate, cervical and melanomas. LO is treatable but it is not curable.

Low Level Laser Therapy (LLLT)

Moderate and high power lasers have been adopted in Western medicine chiefly for their ability to heat tissue to levels that alter tissue structure (e.g. treatment of diabetic retinal neovascularisation; laser surgery); the basis of these laser effects is massive local delivery of photon energy effecting tissue temperature and is not in dispute. However, there is a substantial body of reports in the former Eastern Bloc literature; and also in Western Physiotherapy literature, albeit often poorly controlled, which reports effects of low level laser radiation (LLLT) on cells. A variety of laser wavelengths from visible to near infrared, of diverse powers, application times and treatment regimes, is reported to have inhibitive and stimulative effects at a cellular level (Karu, supra.).

The precise mechanism of how low level laser light affects cells and tissues remains in contention. It has been suggested that laser light interacts with the cytochromes of the mitochondrial electron transport chain.

Many other explanations of laser effects on tissue are offered, including interaction with other cellular elements/processes (porphyrins, cytoskeleton, DNA replication), or marked local gradients in energy delivery due to laser speckle, with resulting local gradients in cellular activity giving stimulation of local cell fluid circulation.

LLLT has been trialed for the treatment of fibrous scar tissue (Thelander and Piller, 2000) and has been shown to affect fibroblasts (Boulton and Marshall, 1986). These effects are important both in treating surgical scars associated with post-mastectomy lymphedema (PML) and in treating the brawny edema that often develops in lymphedematous limbs. It has also been suggested that LLLT encourages lymphogenesis and stimulates lymphatic motoricity (Leivens, 1985; Lievens, 1991). Finally, LLLT is seen to affect macrophage cells (Young et al, 1989) and to stimulate the immune system (Tadakuma, 1993). All of these actions indicate that LLLT could be an appropriate treatment for post-mastectomy lymphedema.

Preliminary evidence using a scanning laser (Piller and Thelander, 1995) showed a beneficial effect when the PML arm and the anterior chest was treated. We sought to apply the laser in the axillary zone, which represents the supposed site of blockage of lymphatic drainage from the limb. We reasoned that the laser may reduce fibrosis and activate surviving lymphatic drainage pathways, stimulate the growth of new pathways, and/or stimulate a localized lymphocyte response that assists in resolving the LO.

Possible explanations for the beneficial effect of LLLT treatment include;

restoration of lymphatic drainage through the axillary region due to stimulation of new lymphatic pathways.

restoration of drainage through reduction of fibrosis and scarring of tissues in the axillary region. There was evidence of tissue softening after LLLT treatment.

systemic effects of LLLT, since the response of the limb occurs despite the laser being applied to tissue which is upstream of the lymphedematous arm. In addition, there also appeared to be changes in extracellular fluid volume in the upper torso and the unaffected limb, sustained for a 1-3 month period after treatment.

reduction in tissue fluid accumulation through changes in blood flow, either directly via an effect of blood vessels or by neural or humoral regulation of vessels in the limb.

increased lymphatic vessel motoricity resulting in increased fluid pumping from the area.

decrease in the widespread fibrotic induration of lymphatic territories, which is associated with chronic low-level inflammatory process in tissues with higher than normal levels of protein in the tissues.

decreased fibrotic induration allowing extracellular fluid (ECF) to move more freely to areas where it can be collected by intact lymphatic vessels (ie. proximal to the site of surgical interruption).

Modification of skin micro-vessel parameters affecting fluid flux across the capillary wall (ie. decreased fluid leakage into the limb).

Further improvements in the use of low level laser in the treatment of a range of conditions rests on a better understanding of its mode of action. The mechanism (s) of action of LLLT in tissues remains elusive, and is complex, likely involving many aspects of tissue physiology. Furthermore, it is dependent on the wavelength, dose, frequency, duration and repeatability of the LLLT applied. At the molecular level, there are reports that LLLT affects cytochromes of the mitochondrial electron transport chain (Karu 1989), and induces local gradients in energy delivery due to laser speckle resulting in local gradients in cellular heating (Horváth & Donko 1992). At the cellular level, LLLT is reported to stimulate mitogenic activity, adhesion, synthetic activity and viability of fibroblasts (Abergel et al, 1984; Boulton & Marshall 1986; Glassberg et al 1988; Yu et al, 1994; Conlan et al, 1996, Bednarska et al, 1998), although this may only be true for systems that are operating sub-optimally (Abergel et al, 1984). Macrophages were stimulated by LLLT to produce factors that increased or decreased fibroblast proliferation, depending on the wavelength of laser used (Young et al, 1990). LLLT stimulate lymphocytes to proliferate and to become activated, both in vitro and in vivo (Inoue et al 1989; Tadakuma, 1993; Ganju et al, 1999), although again this may only be true in pathological settings, where LLLT ‘primes’ lymphocytes to be more responsive to natural stimulatory products (Smol'yaninova et al, 1991). All of these cell types may have a role to play in resolution of lymphedema.

At the microcirculatory level, there may be stimulatory/protective effects of LLLT on endothelial cells and vascular endothelium in situ (Lamuraglia et al 1992). This may involve angiogenic factor production by T-lymphocytes (associated with endothelial cell proliferation; Agaiby et al, 2000), or increased vascular endothelial growth factor (VEGF) production by smooth muscle cells or fibroblasts (Kipshidze et al, 2001). Use of LLLT enhanced endothelial regeneration after damage in animal models (De Scheerder et al, 1998; Kipshidze et al, 1998), and in humans after coronary arterial stent implantation (De Scheerder et al, 2000). We have not found any reports of LLLT on lymphangiogensis, but it is likely that lymphatic vessels will respond similarly to blood vessels, since members of the VEGF family, VEGF-C and -D, stimulate lympangiogensis (Plate, 2001). There are reports of stimulation of local fluid circulation (Horváth & Donko, 1992), and stimulatory effects on lymphatic vessels (Lieviens et al, 1985), probably in response to increased fluid mobility in radiated tissues. There does not seem to be a direct consistent effect of low level laser on lymphatic vessel contractility when laser is applied to the vessels alone (Carati et al, 1998).

Edema (sometimes spelt oedema) is clinically known as the presence of abnormally large amounts of fluid in the intercellular tissue space of the body, usually applied to demonstrable accumulation of excessive fluid in the subcutaneous tissues. Edema may be localised, due to venous or lymphatic obstruction or to increased vascular permeability or it may be systemic due to heart failure or renal disease. Collections for edema fluid are designated according to the site, for example ascites (peritoneal cavity), hydrothorax (pleural cavity) and hydropericardium (pericardial sac). Massive generalised edema is called anasarca.

The invention described herein provides a treatment for edema and lymphedema. The invention in particular will be effective in the treatment of lymphedema that includes the effects of lymph gland damage, disease or removal following surgery typically associated with cancer treatment or at least provides an alternative to other treatments. In particular the use of LLLT can effectively treat LO and post surgery LO of a limb associated with the removal of a lymph gland.

### BRIEF DESCRIPTION OF THE INVENTION

In a broad aspect of the invention, a method of treatment of the lymphatic system in a mammalian subject including the following step:

radiation of the surface of the skin of the mammal in the area of physiological concern with a low level infrared laser.

In a broad aspect of the invention, a method of treatment of lymphedema in a mammalian subject including the following step:

radiation of the surface of the skin of the mammal in the area of physiological concern with a low level infrared laser.

In an aspect of the method the laser is a Class 1 laser (FDA CDRH) Class 1M (EN 60825).

In an aspect of the method the area of physiological concern is the nodal area adjacent to an affected limb.

In a yet further aspect of the invention the laser is a laser arranged to emit a pulsed beam of average output power between 3 and 10 mW.

In another aspect of the invention the wavelength of the laser emission is between 600 to 1100 nm the laser having pulse widths from 1 nanosecond to 1 second with peak powers from 1 milliwatt to 1000 Watts, average powers from 1 microwatt to 1000 milliwatts at repetition rates from 0.1 to 100 kilohertz.

In an embodiment of the invention for treatment of Post Mastectomy LO treatment the laser frequency is 904 nanometres, 2.5 and 5 kilohertz, 200 nanosecond-pulse width, 2.5 and 5 milliWatts average power and 5 Watts peak power.

In another aspect of the invention for Post Mastectomy LO treatment the energy of the laser is delivered at substantially 5 Joules with an energy density of about 1.5 Joules per square centimetre, while maintaining the safety classification of Class 1 (FDA CDRH) Class 1M (EN 60825).

In a further aspect of the invention the laser arrangement uses multiple non-overlapping spots of substantially 5 mm in diameter and about 10 to 20 mm apart.

In a further aspect of the invention, the laser output is radiated on to the mammalian body either directly or via optical transmission fibre.

In yet a further broad aspect the invention is a method of reducing the level of extra-cellular fluid in the tissue of a mammal including the step of radiation of the surface of the skin of the mammal with a low level infrared laser in the vicinity of the area having extra-cellular fluid.

Specific embodiments of the invention will now be described in some further detail with reference to and as illustrated in the accompanying figures. These embodiments are illustrative, and not meant to be restrictive of the scope of the invention. Suggestions and descriptions of other embodiments may be included within the scope of the invention but they may not be illustrated in the accompanying figures or alternatively features of the invention may be shown in the figures but not described in the specification.

### BRIEF DESCRIPTION OF THE FIGURES

FIG. 1 depicts a laser device LTU904H used in the trials;

FIG. 2 depicts the Study Protocol and Treatment Regimen;

FIG. 3 depicts the mean change in affected arm volume immediately after treatment (after Rx), 1 month (mo), or 2 months after treatment. (means±SE);

FIG. 4 depicts the Frequency distribution of individual changes in affected arm volume 2-3 months after treatment;

FIG. 5 depicts the mean change in bio-impedance (arbitrary units) after treatment (after Rx), 1 month (mo), or 2 months after treatment. (means±SE; * p <0.05, ** p<0.01, significantly different from pre-treatment values, ); and

FIG. 6 depicts the frequency distribution of individual changes in extracellular fluid in affected arm 2-3 months after treatment.

### DETAILED DESCRIPTION OF AN EMBODIMENT OF THE INVENTION

The laser device used in the trial was the LTU904H (RianCorp Pty Ltd, Adelaide, Australia). This is a laser which emits in a preferred arrangement a pulsed 904 nm beam, having an average output power emitting 5 Joules with an energy density of about 1.5 Joules per square centimetre, while maintaining the safety classification of Class 1 (FDA CDRH) Class 1M (EN 60825). The laser device is capable of operating between 600-1100 nanometres pulsed at a frequency between 0.5 hz and 100 kilohertz. The radiation can be delivered to the subject either directly or via optical transmission fibre. The laser is also capable of delivering an average power range from 1 microWatt to 1000 milliWatts. A pulsed form of the laser is used as the inventors thought that this would beneficially stimulate and alter the relevant cells as well as penetrate the skin of the patient to reach relevant the area of physiological concern.

The use of a Class 1 device has benefits to the clinician and patient in that the laser is deemed non-harmful to humans so procedures and training are not as rigorous as if the laser had a higher classification. Furthermore the laser device is typically cheaper to purchase, maintain and or replace. The laser device is generally more reliable in any event because the active device and the control electronics are simple and good design practice will ensure high Mean Time Between Failure.

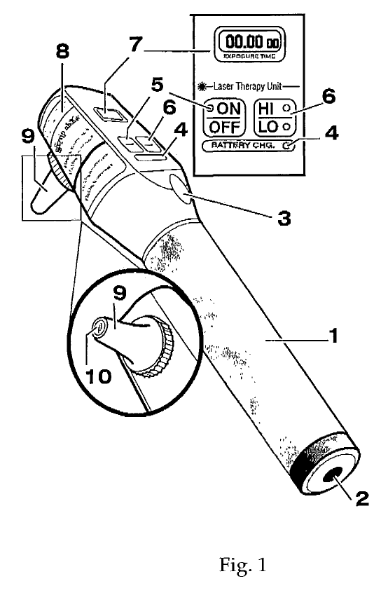

The various elements of the laser device, as depicted in FIG. 1, are as follows:

1: One-piece body.

2: Socket for battery charger connector.

3: Treatment control switch (touch-sensitive, pressure-less type)

4: Battery charging indicator lamp.

5: ON/OFF switch with indicator lamp (membrane-type switch).

6: HI/LO—High output (5 milliwatt) and low output (2.5 milliwatt) selection switch and indicator lamps for each setting (membrane type switch).

7: Elapsed treatment time.

8: Warning labels.

9: Probe head.

10: Transmission window.

The laser device depicted radiates a single beam however it is possible to use a laser arrangement that uses multiple non-overlapping spots of substantially 5 mm in diameter and about 10 to 20 mm apart.

Method:

A prospective, double blinded, placebo controlled, randomized, single crossover trial is used to illustrate the efficacy of the method of treatment of LO. All patients attending, or newly presenting to, the Flinders Medical Center Lymphoedema Assessment Clinic (Flinders Medical Center, South Australia) was considered for entry into the trial. The trial was conducted over a 24 month period, with data randomly collected for each group through all sessions.

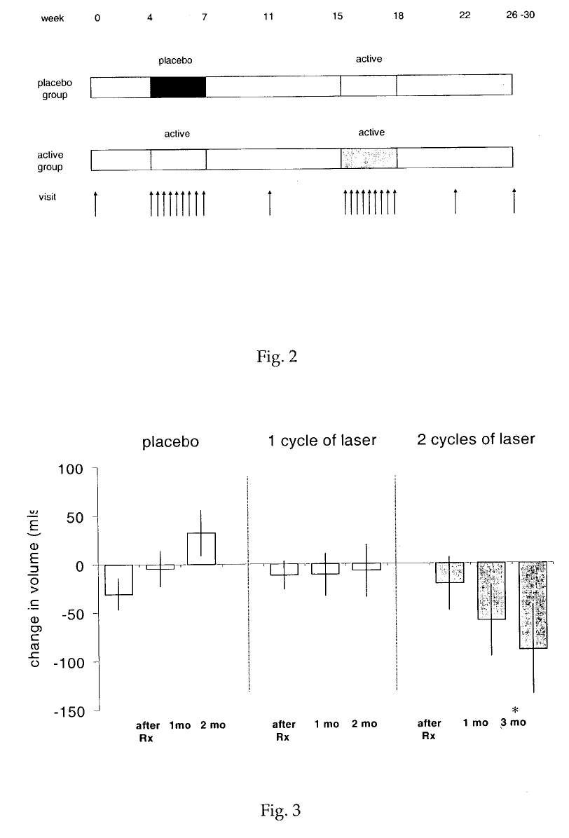

The trial was designed to allow comparisons between placebo treatment, one cycle of LTU904H, or two cycles of LTU904H, and to ensure that all participants received at least one cycle of LTU904H treatment. Participants were allocated into the ‘active’ or ‘placebo’ group using a random number table. Those participants entering the ‘placebo’ group received 1 block of sham therapy, followed by an 8 week rest period, then 1 block of active LLLT (FIG. 2). The ‘active’ group received 2 blocks of LLLT, separated by an 8 week rest period. Since statistical analysis (see Results Section) showed no ongoing effect from placebo treatment, some ‘placebo’ participants were then offered a 2^{nd} block of active laser therapy and these results were included in the analysis of active treatment.

Patient Selection:

A standard procedure was used to screen patients for inclusion. The following criteria had to be met before a patient was entered into the trial:

Age—at least 18 years;

Sex—female only;

Diagnosis—clinically manifest PML (>200 ml difference between arms or ≧2 cm difference in arm circumference at ≧3 positions);

Administrative—the patient understood the trial and was able to provide informed consent.

Participants were excluded on the following criteria:

Presence of certain comorbidities—current metastases, history of severe trauma/disruptive surgery to the arm;

Instability of condition—significant changes to the arm in the past 3 months, including change in treatment regime or occurrence of cellulitis;

Clinical—inability to abduct arm sufficient for measuring purposes;

Diagnosis—presence of primary lymphedema in the lower limbs.

Study Protocol:

Treatment was delivered in blocks of 9 sessions (active laser or placebo), where 1 block consists of treatment 3 times per week for 3 weeks. A grid was designed to sit in the axilla with treatment points marked at 2 cm intervals to guide application. Each treatment point was treated for 1 minute and there were a total of 17 points, making the treatment time 17 minutes per session. The laser was held in contact with, and at right angles to, the skin. The total energy applied was 5.1 Joules at a dosage of 1.53 J/cm² (see FIG. 3).

Patient Assessment

Objective measures were taken at the start, and at the end of the LTU904 treatment, of every visit, as follows;

1. Perometry uses infrared sensors to measure the limb circumference at every 4 mm's, with limb volume calculated via a truncated cone method (Perometer 350S and Pero Plus v1.4 software, Perosystem Meβgerat, Wuppertal, Germany). This is regarded as a very accurate assessment of limb volume (Stanton et al, 1997).

2. Bio-impedance measures electrical impedance to alternating electrical currents (100 μA, 50 kHz), thereby giving an objective measure of extra-cellular fluid (ECF) levels in various parts of the body (Cha et al, 1997; Lee et al, 2001). We used a Inbody 3.0 system (Biospace, Korea), which provides whole body, trunk, torso and limb ECF values (Cha et al, 1997). Body weight and mass index were also monitored using the Inbody 3.0 system.

3. Tonometry measures tissue resistance to pressure, giving an indication of the compliance of the dermis and extent of fibrotic induration in a limb (Clodius et al, 1976). The tonometer (COMPAC, Switzerland) consists of a central plunger (1 cm diameter) weighted to a mechanical load of 275.28 gms/cm², operating through a footplate which rests on the surrounding skin and applies a load of 12.2 gms/cm². Thus, the plunger applies a differential pressure of 263 gms/cm², and the degree of penetration of the plunger (arbitrary units) is measured by a micrometer.

Tonometry of the upper and lower affected and unaffected arm, and the anterior and posterior torso was measured,

1. Shoulder range of movement (ROM) was assessed using a goniometer (Jamar, Miami, USA)

Data Analysis

Data were analysed using SPSS version 10.55 or 11 (SPSS Inc, USA) using analysis of variance and multiple regression. Comparisons were made between or within participant groups receiving placebo only, or one or two cycles of active laser treatment. Significance (at p<0.05) was determined by Student's T-test or Fischer exact tests for comparisons between groups; comparisons within groups were by paired t-tests. To assess the change in any parameter, the mean of the first two visits were subtracted as a baseline measurement. Power analysis was performed using nQuery.

Results

Twenty seven participants entered the 'placebo’ group, and 26 participants entered the ‘active’ group. Preliminary statistical analysis showed that there were no significant differences between participants who received one cycle of active laser treatment in the ‘placebo’ group compared those receiving the first cycle of active treatment in the ‘active’ group. That is the placebo treatment did not affect the outcome of a single cycle of active laser treatment). Consequently, 11 participants from the ‘placebo’ group chose to have a 2^{nd} cycle of 3 weeks of active laser therapy, making a total of 37 participants who had 2 cycles of active laser therapy following the ‘active’ protocol. In all, 64 participants (27 ‘placebo’ group and 37 in ‘active’ group) completed the trial. Of these, 26 and 29 were available for three month follow-up, respectively.

Effect of LTU904 Treatment on Arm Volume.

There was no significant effect of placebo treatment only, or one cycle of laser treatment only, on mean affected limb volume (Table 1, FIG. 3). Mean affected limb volume was not significantly reduced immediately after 2 cycles of active laser treatment (p=0.442), but continued to decrease at one (p=0.119) or three month (p=0.62) follow-up after the cessation of treatment. Change in volume at 3 months after two cycles of treatment was significantly less than after placebo treatment (p=0.017).

TABLE 1
Mean change in affected arm volume
(− means reduction; mean ± standard error)

| | Immediately after | One month after | 2-3 months after |
|---|---|---|---|
| | treatment | treatment | treatment |
| Placebo | −30.4 ± 16.2 | −4.9 ± 18.4 | 32.1 ± 23.4 |
| One cycle of | −11.6 ± 14.8 | −11.3 ± 21.7 | −7.5 ± 27.1 |
| active | | | |
| treatment | | | |
| Two cycles of | −21.1 ± 27.2 | −59.2 ± 37 | −89.7 ± 46 |
| active | | | |
| treatment | | | |

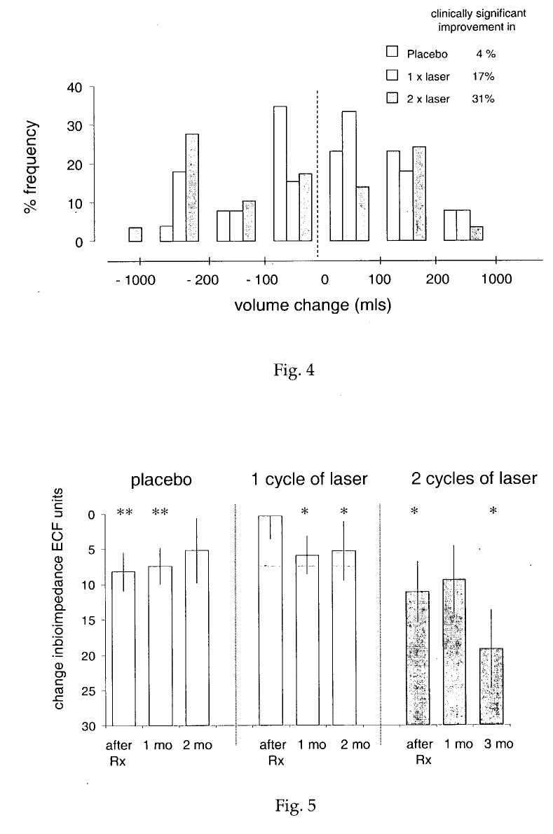

The criteria for success for individuals was defined as a decrease of 200 mls in LO affected limb volume (Table 2). Successful long term effectiveness of LTU904 treatment was defined by a 200 ml reduction in limb volume (from initial measure) maintained in the months after cessation of treatment. There were no significant differences in this criterion between treatments immediately after cessation of the treatment. However, both one and two cycles of treatment were significantly better than placebo treatment after one month, and two cycles of treatment were significantly better than one cycle of treatment after two-three months (FIG. 4). Thirty one % of subjects had a clinically significant reduction in their LO affected arm two-three months after treatment with 2 cycles of LTU904 treatment (significantly better than placebo, Fischer's exact test, p≧0.05).

TABLE 2
Number of patients showing a ≧200 ml reduction in arm volume

| | Immediately after | One month after | 2-3 months after |
|---|---|---|---|
| | treatment | treatment | treatment |
| Placebo | 7.4% (2 of 27) | 0% | 3.8% (1 of 26) |
| One cycle of | 4.5% (2 of 44) | 12.2% (5 of 41) | 17.9% (7 of 39) |
| active | | | |
| treatment | | | |
| Two cycles of | 10.8% (4 of 37) | 17.1% (6 of 35) | 31% (9 of 29) |
| active | | | |
| treatment | | | |

Effect of LTU904 Treatment on Extracellular Fluid (ECF) Distribution

Extracellular fluid (ECF) was measured using arbitrary bio-impedance units; an increase in these units indicates a decrease in extracellular fluids.

ECF of both the affected (FIG. 5) and unaffected arm was significantly reduced by placebo or one cycle of LTU904H treatment. However, ECF was most significantly reduced following 2 cycles of LTU-904H therapy, in the following regions;

the affected arm (immediately after the course of treatment (p=0.014, paired t-tests) and maintained at 1 month (p=0.027) and 3 month follow-up (p=0.017; FIG. 5)

the unaffected arm.(immediately after treatment (p=0.009) and maintained at 3 month follow-up (p=0.042)).

AND

the trunk (at 1 month (p=0.027) and 3 month (p=0.040)) follow-up

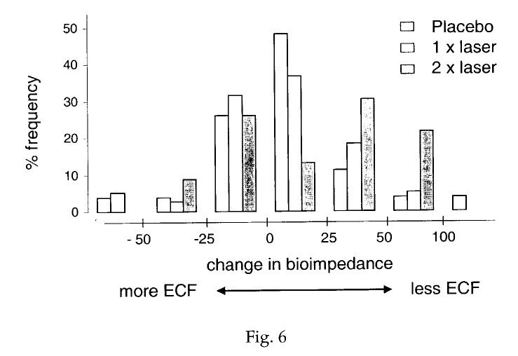

A greater proportion of participants showed reductions of ECF of the affected arm at 2-3 months after 2 cycles of LTU904 treatment, compared to one cycle or placebo treatment. 52% of participants receiving 2 cycles of treatment had changes in bio-impedance of 25 or more, compared to 23% and 24% receiving 1 cycle or placebo respectively (FIG. 6).

Effect of LTU904 Treatment on Tonometry

Tonometry assesses the ‘hardness’ of the tissue, and is an index of fibrotic induration. The lower the tonometry reading, the ‘harder’ the tissue.

If untreated, lymphedema causes hardening of the limb over time. There were significant decreases in tonometry (indicating increased tissue ‘hardness’) in participants receiving placebo or one cycle of LTU904H treatment over the duration of the trial. Participants in the ‘active’ group tended to have softening of the tissues (as measured by increased tonometry readings).

There were significant ‘hardening’ of the affected arm and torso immediately after treatment with 2 cycles of LTU904H, but at 3 months after treatment there was a significant increase in tissue tonometry (indicating softening of the tissues) in the affected upper arm (p=0.025).

Effect of LLLT Treatment on Range of Movement.

There was no consistent effect of any treatment of Range of Movement of the affected arm.

The inventors conclude that LLLT treatment according to the method described herein improved the condition of the lymphedema (PML) affected limbs of participants in the trial, as assessed by a number of criteria. Most significant was the clinically robust reduction in limb volume of ≧200 mls for a period of 3 months or more in 30% of participants compared to 3.8% in the placebo group. This finding was corroborated by similarly sustained reductions in extracellular fluid of the affected arm and torso region, and the improvements in tissue ‘hardness’. Whilst no one parameter measured is definitive of successful treatment of PML, taken together they suggest LLLT treatment is a promising approach to the resolution of lymphedema. Two cycles of LLLT were better than one cycle of treatment, which was not much better than placebo. Effects of LTU904H take sometime to develop, and were sustained for up to 3 months after LLLT treatment.

In conclusion, two cycles of LLLT treatment was effective in reducing whole arm volume, extra-cellular fluid, and fibrotic induration in post-mastectomy lymphedema in 31% of participants at 3 month follow-up after treatment.

References

Agaiby, A, Ghali, L R, Wilson, R and Duson, M (2000). Laser modulation of angiogenic factors production by T-lymphocytes. Lasers Surg. Med. 26;357-363.

Boulton, M. & Marshall, J. (1986). He-Ne laser stimulation of human fibroblast proliferation and attachment in vitro. Lasers in Life Sci., 1; 125-134

Browning C. (1997) Lymphoedema: prevalence, risk factors and management: a review of research. Sydney: NHMRC National Breast Cancer Centre.

Carati C J, Jobling, J, Fouyaxis, J, and Gannon B J (1998). Effect of low level laser on mesenteric lymphatics and blood vessels in-vivo. 2nd Oedema Conference, Australian Lymphoedema Assocaition, Brisbane, March

Casley-Smith, J R and Casley-Smith, J R (1997). The cost-efficiencies of various treatments for lymphoedema. In Modern Treatment for Lymphoedema, Chapt 42; 296-304. The Lymphoedema Association of Australia, Adelaide South Australia. ISBN 0646316648

Cha, K, Chertow, G M, Gonzalez, J, Lazarus, J M and Wilmore, D W (1995) Multifrequency bioelectrical impedance estimates the distribution of body water. J Appl. Physiol., 79 (4); 1316-1319

Clodius, L, Deak, L and Piller, N B (1996) A new instrument for the evaluation of tissue tonicity in lymphoedema. Lymphology 9; 1-5

Conlan, M J, Rapley, J W and Cobb, C M (1996). Biostimulation of wound healing by low energy irradiation; a review. J. Clin. Periodontol. 23;492-496.

De Scheerder, I K, Wang, K, Zhou, X R, Verbeken, E, Keelan, M H, Horn, J B, Sahbota, H. and Kipshidze, N (1998). Intravascular low power red laser light as an adjunct to coronary stent implantation evaluated in a porcine coronary model. J. Invas. Cardiol. 10;263-286.

De Scheerder, I K, Wang, K, Zhou, X R, Szilard, M, Verbeken, E, Ping, Q B, Yanming, H, Jianhua, H and Van der Werf, F (2000). Intravascular low power red laser light as an adjunct to coronary stent implantation: initial clinical experience. Cathet. Cardiovasc. Intervent. 49; 468-471.

Foldi E, Foldi M, Weissleder H. (1985) Conservative treatment of lymphedema of the limbs. Angiology, March; 171-180.

Ganju, L, Salhan,A, Dev Karan, Chanda, S and Srivastara, K K (1999). Immunomodulatory effect of laser on whole body exposure. Indian J Exper. Biol. 37;444-449

Glassberg, E, Lask, G P, Tan, E M and Uitto, J (1988). Cellular effects of the tunable pulsed dye laser at 577 nanmeters on human endothelial cells, fibroblasts and erythrocytes; an in vitro study. Lasers. Surg. Med. 8:567-72.

Horvath, Z G and Donko, Z (1992). Possible ab-inition explanation of laser <<biostimulation>> effects. In Laser Applications in Medicine and Surgery, Galletti, G. et al eds, Monduzzi, Bollogna; 57-60.

Inoue, K, Nishioka, J and Hukuda, S (1989). Altered lymphocyte proliferation by low dosage laser irradiation. Clin. Exp. Rheumatol. 7; 521-523.

Karu, T. (1989). Photobiology of low power laser effects. Health Physics. 56; 691-704

Kipshidze, N, Sabota, H, Komorowski, R, Nikolaychik, V, and Keelan, M H (1998). Photoremodelling of arterial wall reduces restenosis following balloon angioplasty in an artheroschlerotic rabbit model. J. Am. Coll. Cardiol. 31; 1152-1157

Kipshidze, N, Nikolaychik, V, Keelan, M H, Shankar, L R, Khanna, A Komorowski, R, Leon, M and Moses, J. (2001). Low power Helium-Neon laser irradiation enhances production of vascular endothelial growth factor and promotes growth of endothelial cells in vitro. Lasers. Surg. Med. 28:355-364.

LaMuraglia, G M et al (1992) Photodynamic application on arteries. In Laser Applications in Medicine and Surgery, Galletti, G. et al eds, Monduzzi, Bollogna; 439-444.

Lee, S W, Song, J H, Kim, G A, Lee, K J and Kim, M J (2001) Assessment of total body water from the anthropometry-based equations using bioelectrical impedance as a reference in Korean adult control and hemodialysis subjects. Nephrol. Dial. Transplant. 16: 91-97.

Lievens P C. (1991) The effect of combined HeNe and I.R. laser treatment on the regeneration of the lymphatic system during the process of wound healing. Lasers in Medical Science 6; 193-199.

Lievens P C. (1985), The influence of laser-irradiation on the motoricity of the lymphatical system and on the wound healing process. Proc International Congress on Laser in Medicine and Surgery; 171-174.

Plate, K H (2001). From angiogenesis to lymphangiogenesis. Nat. Med. 7(2);151-152.

Petrek J A and Heelan M C. (1998). Incidence of breast carcinoma-related lymphedema. American Cancer Society 83; 2776-2781.

Piller, N. B & Thelander, A. (1995) Treating chronic post mastectomy lymphoedema with low level laser therapy: a cost effective strategy to reduce lymphoedema severity and improve the quality of patient life. Lymphology, 31, 74-86.

Smol'yaninova, N K, Karu, T I, Fedoseeva,G E and Zelinin, A V (1991). Effects of He—Ne laser irradiation on chromatin properties and synthesis of nucleic acids in human peripheral lymphocytes. Biomedical Sci 2;121-126.

Stanton, A W B, Northfield, J W, Holroyd B, Mortimer P S and Levick, J R (1997). Validation of an optoelectronic limb volumeter (Perometer). Lymphology 30; 77-97.

Tadakuma, T (1993) Possible application of the laser in immunobiology. Keio J Med 42 (4): 180-182

Thelander A and Piller N B. (2000) Post surgical oedema treated by low level scanning laser. Third Australasian Lymphology Association Conference (2000), 129-132.

Tobin M B, Lacey H J, Meyer L, Mortimer P S (1993). The psychological morbidity of breast-cancer related arm swelling. Psychological morbidity of lymphoedema. Cancer 72; 3248-3252.

Young S, Bolton P, Dyson M, Harvey W and Diamantopoulos C. (1989), Macrophage responsiveness to light therapy. Lasers in Surgery and Medicine 9; 497-505.

Yu, W, Naim, J O, and Lanzafame, R J (1994). The effect of laser irradiation on the release of bFGF from 3T3 fibroblasts. Photochem. Photobiol 59;167-170.

It will be appreciated, by those skilled in the art, that the invention is not restricted in its use to the particular application described. Neither is the present invention restricted in its preferred embodiment with regard to the particular elements and/or features described or depicted herein. It will be appreciated that various modifications can be made without departing from the principles of the invention. Therefore, the invention should be understood to include all such modifications within its scope.

The claims defining the invention are as follows:

1. A method of treatment of the lymphatic system in a mammalian subject including the following step:
radiation of the surface of the skin of the mammal in the area of physiological concern with a low level infrared laser.

2. A method in accordance with claim 1 for the treatment of lymphedema in a mammalian subject including the following step:
radiation of the surface of the skin of the mammal in the area of physiological concern with a low level infrared laser.

3. A method in accordance with claim 1 wherein the laser is a Class 1 laser (FDA CDRH) Class 1M (EN 60825).

4. A method in accordance with claim 3 wherein the laser is a laser arranged to emit a pulsed beam of average output power between 3 and 10 mW.

5. A method in accordance with claim 1 wherein the area of physiological concern is the nodal area adjacent to an affected limb.

6. A method in accordance with claim 1 wherein the wavelength of the laser emission is between 600 to 1100 nanometers having pulse widths from 1 nanosecond to 1 second, peak powers from 1 milliwatt to 1000 Watts and average powers from 1 microwatt to 1000 milliwatts at repetition rates from 0.1 to 100 kilohertz.

7. A method in accordance with claim 6 for Post Mastectomy LO treatment wherein the laser frequency is 904 nanometres, 2.5 and 5 kilohertz, 200 nanosecond pulse width, 2.5 and 5 milliwatts average power and 5 Watts peak power.

8. A method in accordance with claim 2 for Post Mastectomy LO treatment wherein the energy of the laser is delivered at substantially 5 Joules with an energy density of about 1.5 Joules per square centimetre, while maintaining the safety classification of Class 1 (FDA CDRH) Class 1M (EN 60825).

9. A method in accordance with claim 8 using multiple non-overlapping laser spots of substantially 5 mm in diameter and about 10 to 20 mm apart.

10. A method in accordance with claim 1 wherein the laser output is radiated on to the mammalian body either directly or via optical transmission fibre.

11. A method of treatment of edema in a mammalian subject including the following step:
radiation of the surface of the skin of the mammal in the area of physiological concern with a low level infrared laser.

12. A method in accordance with claim 11 wherein the laser is a Class 1 laser (FDA CDRH) Class 1M (EN 60825).

13. A method in accordance with claim 12 wherein the laser is a laser arranged to emit a pulsed beam of average output power between 3 and 10 mW.

14. A method in accordance with claim 11 wherein the wavelength of the laser emission is between 600 to 1100 nanometers having pulse widths from 1 nanosecond to 1 second, peak powers from 1 milliwatt to 1000 Watts, and average powers from 1 microwatt to 1000 milliWatts at repetition rates from 0.1 to 100 kilohertz.

15. A method in accordance with claim 14 using multiple non-overlapping laser spots of substantially 5 mm in diameter and about 10 to 20 mm apart.

16. A method in accordance with claim 11 wherein the laser output is radiated on to the mammalian body either directly or via optical transmission fibre.

17. A method of reducing the level of extra-cellular fluid in the tissue of a mammal including the step of:
radiation of the surface of the skin of the mammal with a low level infrared laser in the vicinity of the area having extra-cellular fluid.
